(19) United States
(12) Patent Application Publication
(10) Pub. No.: US 2011/0290902 A1
(43) Pub. Date: Dec. 1, 2011

(54) ATMOSPHERIC DIFFUSER APPARATUS

(75) Inventor: Phyllis Rubel, Westfield, NJ (US)

(73) Assignee: PIC Corp., Linden, NJ (US)

(21) Appl. No.: 12/790,446

(22) Filed: May 28, 2010

(51) Int. Cl. A61L 9/04

(52) U.S. Cl. 239/34

(57) ABSTRACT

An insect repellent scent diffusing apparatus including a container having a bottom wall and a continuous side wall surface extending from the bottom wall. A plurality of apertures are formed in the continuous side wall surface. An element containing insect repellent scent is disposed in the container and configured to diffuse insect repellent scent through the plurality of apertures and into the surrounding atmosphere.

### FIELD OF THE INVENTION

The invention relates to dispensing apparatus, and more particularly to apparatus for dispensing insecticide into the atmosphere.

### BACKGROUND OF THE INVENTION

An insect repellent is a substance which may come in many forms and applications, including being applied to skin, clothing, or other surfaces which discourages insects (and arthropods in general) from landing or climbing on that surface. Insect repellents help prevent and control the outbreak of insect-borne diseases such as malaria, Lyme disease, Dengue fever, bubonic plague, and West Nile fever. Pest animals commonly serving as vectors for disease include the insects flea, fly, and mosquito; and the arachnid tick. Usually insect repellents work by masking human scent, or by using a scent which insects naturally avoid. Common insect repellents include: DEET; Essential oil of the lemon eucalyptus and its active compound p-menthane-3,8-diol (PMD); Icaridin, also known as picaridin, Bayrepel, and KBR 3023; Nepetalactone, also known as “catnip oil”; Citronella oil; Permethrin; Neem oil; and Bog Myrtle

A know prior art method of diffusing insect repellents into the atmosphere was through allowing diffusion of vapor or smoke into the surroundings of the device, and particularly, but not exclusively, to devices allowing diffusion of dry smoke produced from a combustible solid.

It is known to use a diffusion to burn incense in order to introduce fragrance into a room. The introduction of fragrance into a room can be a mood enhancer and can also mask unwanted household odors such as those caused by cooking, tobacco, pets, etc.

It is also known to use a diffusion to burn solid blocks of combustible material, incorporating either fragrance or other additives such as insecticides etc. However, known diffusion methods suffer from problems relating to heat transfer from the diffusion to the surface on which the diffuser is positioned, and inefficient burning of the solid block resulting in sub-optimal burning of the solid block. Further, with known diffusion it is common for the ash from the burnt solid block to spill over from the diffusion onto a surface on which the diffuser is positioned.

Other known methods for diffusing or dispersing insect repellents into the atmosphere include: aerosol vapor delivery systems (e.g., OFF™ spray); smoke barriers in which flames originating from a material are extinguished whereafter the material continues to generate smoke which acts as an insect deterrent (e.g., burning mosquito coils); using open flames (e.g., burning citronella candles); battery and/or electric operated devices configured and operable to attract or repel insects; and skin lotions having insect repellent qualities.

### SUMMARY OF THE INVENTION

In one aspect, an apparatus for diffusing insect repellent scents into the atmosphere in accordance with certain illustrated embodiments is disclosed. It is noted, some of the advantages provided by the present invention in accordance with the illustrated embodiments include; no open flame is used (which obviates the accompanying fire hazard and/or potential for skin burn); no aerosols are used (which obviates the use of accompanying air pollutants); no lotions need to be applied to skin; and there are no associated operational costs.

The apparatus includes a container having a bottom container member defining a bottom wall and a continuous side wall surface extending from the bottom wall. The bottom container member has a first outer surface and a second outer surface extending from the first outer surface. The second outer surface forms a top portion of the bottom container member. A plurality of apertures are defined in the top portion of the second outer surface for diffusing insect repellent scents.

The container further includes a top lid member configured to detachably attach to the top portion of the bottom container wherein the first outer surface of the bottom container member has a circumference substantially identical to that of the outer surface of the top lid member and the second outer surface of the bottom container member has a circumference which is less than that of the outer surface of the top lid member such that the top lid member slideably receives on the second outer surface of the bottom container member. The top lid member is movable from a first position wherein all of the apertures of the bottom container member are concealed to a second position in which at least some of the apertures of the bottom container member are exposed when abutting portions of respective patterns provided on the top lid member and the bottom container member are caused to abut one another.

The container further includes an element disposed in the container and configured to diffuse insect repellent scent through the exposed apertures and into the surrounding atmosphere when the top lid member is in the second position.

### BRIEF DESCRIPTION OF THE DRAWINGS

The objects and features of the invention can be understood with reference to the following detailed description of an illustrative embodiment of the present invention taken together in conjunction with the accompanying drawings in which:

FIG. 1 is an exploded view of the present invention in accordance with an illustrated embodiment;

FIG. 2 depicts the present invention of FIG. 1 in a closed position; and

FIG. 3 depicts the present invention of FIG. 3 in an open position.

### WRITTEN DESCRIPTION OF CERTAIN EMBODIMENTS OF THE INVENTION

The present invention is now described more fully with reference to the accompanying drawings, in which illustrated embodiments of the present invention are shown. The present invention is not limited in any way to the illustrated embodiments as the illustrated embodiments described below is merely exemplary of the invention, which can be embodied in various forms, as appreciated by one skilled in the art. Therefore, it is to be understood that any structural and functional details disclosed herein are not to be interpreted as limiting, but merely as a basis for the claims and as a representative for teaching one skilled in the art to variously employ the present invention. Furthermore, the terms and phrases used herein are not intended to be limiting but rather to provide an understandable description of the invention.

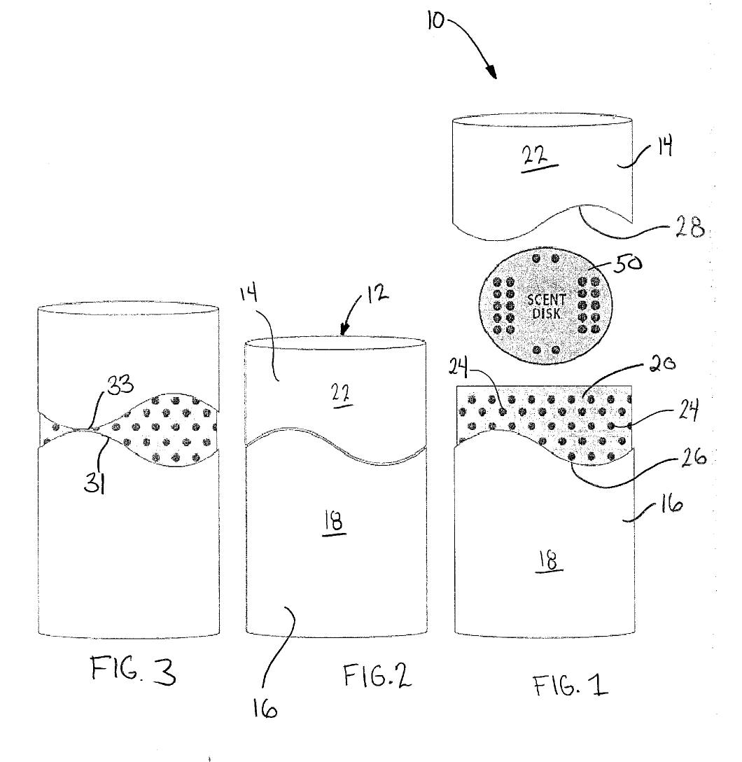

Turning now descriptively to the drawings, in which similar reference characters denote similar elements throughout the several views, FIG. 1 depicts an insect repellent diffusion delivery system, designated generally by reference numeral 10, in accordance with the certain illustrated embodiments of the invention. Delivery system 10 includes a container 12 having a top lid member 14 and a bottom container member 16 each configured to detachably fit with one another to form the container 12 depicted in FIG. 2. The top lid member 14 and bottom container member 16 are preferably cylindrically shaped and each has a closed bottom wall surface and an opposing open top surface, as illustrated. However, container 10 is not to be understood to be limited to having a cylindrical shape as it may be configured to have numerous other shapes.

Preferably, an element 50 configured to emit (or diffuse) an insect repellent scent, is removably disposed in the bottom container member 16. The element 50 depicted in FIG. 1 is shown in the shape of a disc. However, it is no to be understood to be limited thereto as it may encompass any form or shape capable of emitting (or diffusing) an insect repellent scent that may be disposed within the container 12. For instance, the element can be in the form of a liquid or the interior surface portion of one of, or both, the top lid member 14 and bottom container member 16 is infused, impregnated, saturated or coated with an insect repellent scent.

As depicted in FIG. 1, the bottom container member 16 is configured to have a first outer surface 18 and a second outer surface 20 extending from the first outer surface 18. The second outer surface 20 forms the top portion of the bottom container member 16. It is to be appreciated that the first outer surface 18 preferably has a circumference which is substantially identical to that of the outer surface 22 of the top lid member 14, and the second outer surface 20 of the bottom container member 16 has a circumference which is less than that of the outer surface 22 of the top lid member 14.

A top portion of the second outer surface 20 of the bottom container member 16 is provided with a plurality of apertures 24. Additionally, the first outer surface 18 of the bottom container member 16 defines a serpentine or wavelike like pattern 26 along its upper surface. Likewise, the bottom surface 28 of the top lid member 14 is also preferably formed with a serpentine or wave like pattern 28 similar to that of the serpentine or wave pattern 26 of the first outer surface 18 of the bottom container member 16.

As shown in FIG. 2, when the top lid member 14 is fitted to the bottom container member 16, and their respective aforesaid serpentine or wave like patterns 26, 28 are caused to align with one another, each of the plurality of apertures 24 of the bottom container member 16 are concealed by the top lid member 14. Thus, the container 10 is to be understood to be in a closed position preventing vapor scents from disc-like element 50 to diffuse thereform.

In contrast, and with reference to FIG. 3, when a user causes the top lid member 14 to rotate or move relative the bottom container member 16 causing protruding portions 31, 33 of the serpentine or wave like patterns 26, 28 respectively on the top lid member 14 and the bottom container member 16 to abut one another, at least some of the plurality of apertures 24 of the bottom container member 16 are exposed thus enabling insect repellent scents to be diffused from the aforesaid disc-like element 50 and into the surrounding atmosphere, via the plurality of apertures 24.

It is to be also appreciated that the aforesaid described embodiments of the present invention, when not is use, may be placed in a scent retaining bag to further preserve the element used containing the insect repellent scent. And when an embodiment contains a liquid element, an air tight lid or seal member is to be used to prevent any unintended evaporation of the liquid element.

Optional embodiments of the present invention may also be said to broadly consist in the parts, elements and features referred to or indicated herein, individually or collectively, in any or all combinations of two or more of the parts, elements or features, and wherein specific integers are mentioned herein which have known equivalents in the art to which the invention relates, such known equivalents are deemed to be incorporated herein as if individually set forth.

Although illustrated embodiments of the present invention has been described, it should be understood that various changes, substitutions, and alterations can be made by one of ordinary skill in the art without departing from the scope of the present invention.

What is claimed is:

1. An apparatus for diffusing insect repellent scents into the atmosphere, the apparatus comprising:
a container comprising:
a bottom container member having a bottom wall and a continuous side wall surface extending from the bottom wall wherein a plurality of apertures are formed in a top portion of the continuous side wall surface; and
a top lid member configured to detachably attach to the top portion of the bottom container and movably from a first position wherein all the apertures of the bottom container member are concealed to a second position in which at least some of the apertures of the bottom container member are exposed; and
an element disposed in the container and configured to diffuse insect repellent scent through the exposed apertures and into the surrounding atmosphere when the top lid member is in the second position.

2. An apparatus for diffusing insect repellent scents as recited in claim 1 wherein the element is in the form of liquid.

3. An apparatus for diffusing insect repellent scents as recited in claim 1 wherein the element is in the form of a solid disc.

4. An apparatus for diffusing insect repellent scents as recited in claim 1 wherein the continuous side wall surface of the bottom container member and the top lid member are cylindrically shaped.

5. An apparatus for diffusing insect repellent scents as recited in claim 1 wherein the bottom container member has a first outer surface and a second outer surface extending from the first outer surface wherein the second outer surface forms a top portion of the bottom container member.

6. An apparatus for diffusing insect repellent scents as recited in claim 5 wherein the first outer surface of the bottom container member has a circumference substantially identical to that of an outer surface of the top lid member and the second outer surface of the bottom container member has a circumference which is less than that of the outer surface of the top lid member such that the top lid member slideably receives on the second outer surface of the bottom container member.

7. An apparatus for diffusing insect repellent scents as recited in claim 6 wherein the first outer surface of the bottom container member defines a pattern along its upper surface and a bottom surface of the top lid member defines a pattern similar to that of the pattern of the first outer surface of the bottom container.

8. An apparatus for diffusing insect repellent scents as recited in claim 7 wherein the top lid member is positioned in the second position when abutting portions of the respective patterns on the top lid member and the bottom container member are caused to abut one another.

9. An apparatus for diffusing insect repellent scents as recited in claim 7 wherein the respective patterns of the top lid member and the bottom container member are configured in a wave like pattern.

10. An apparatus for diffusing insect repellent scents into the atmosphere, the apparatus comprising:
a container comprising:
a bottom container member a bottom wall and a continuous side wall surface extending from the bottom wall wherein the bottom container member has a first outer surface and a second outer surface extending from the first outer surface wherein the second outer surface forms a top portion of the bottom container member, a top portion of the second outer surface defines a plurality of apertures; and
a top lid member configured to detachably attach to the top portion of the bottom container wherein the first outer surface of the bottom container member has a circumference substantially identical to that of an outer surface of the top lid member and the second outer surface of the bottom container member has a circumference which is less than that of the outer surface of the top lid member such that the top lid member slideably receives on the second outer surface of the bottom container member and is movable from a first position wherein all the apertures of the bottom container member are concealed to a second position in which at least some of the apertures of the bottom container member are exposed; and
an element disposed in the container and configured to diffuse insect repellent scent through the exposed apertures and into the surrounding atmosphere when the top lid member is in the second position.

11. An apparatus for diffusing insect repellent scents as recited in claim 10 wherein the element is in the form of liquid.

12. An apparatus for diffusing insect repellent scents as recited in claim 10 wherein the element is in the form of a solid disc.

13. An apparatus for diffusing insect repellent scents as recited in claim 10 wherein the continuous side wall surface of the bottom container member and the top lid member are cylindrically shaped.

14. An apparatus for diffusing insect repellent scents as recited in claim 10 wherein the first outer surface of the bottom container member defines a pattern along its upper surface and a bottom surface of the top lid member defines a pattern similar to that of the pattern of the first outer surface of the bottom container.

15. An apparatus for diffusing insect repellent scents as recited in claim 14 wherein the top lid member is positioned in the second position when abutting portions of the respective patterns on the top lid member and the bottom container member are caused to abut one another.

16. An apparatus for diffusing insect repellent scents as recited in claim 17 wherein the respective patterns of the top lid member and the bottom container member are configured in a wave like pattern.

17. An apparatus for diffusing insect repellent scents into the atmosphere, the apparatus comprising:
a container comprising:
a bottom container member having a bottom wall and a continuous side wall surface extending from the bottom wall wherein the bottom container member has a first outer surface and a second outer surface extending from the first outer surface wherein the second outer surface forms a top portion of the bottom container member, a top portion of the second outer surface defines a plurality of apertures; and
a top lid member configured to detachably attach to the top portion of the bottom container wherein the first outer surface of the bottom container member has a circumference substantially identical to that of an outer surface of the top lid member and the second outer surface of the bottom container member has a circumference which is less than that of the outer surface of the top lid member wherein the first outer surface of the bottom container member defines a pattern along its upper surface and a bottom surface of the top lid member defines a pattern similar to that of the pattern of the first outer surface such that the top lid member slideably receives on the second outer surface of the bottom container member and is movable from a first position wherein all the apertures of the bottom container member are concealed to a second position in which at least some of the apertures of the bottom container member are exposed when abutting portions of the respective patterns on the top lid member and the bottom container member are caused to abut one another; and
an element disposed in the container and configured to diffuse insect repellent scent through the exposed apertures and into the surrounding atmosphere when the top lid member is in the second position.

18. An apparatus for diffusing insect repellent scents as recited in claim 17 wherein the element is in the form of liquid.

19. An apparatus for diffusing insect repellent scents as recited in claim 17 wherein the element is in the form of a solid disc.

20. An apparatus for diffusing insect repellent scents as recited in claim 17 wherein the continuous side wall surface of the bottom container member and the top lid member are cylindrically shaped.
